(19) United States
(12) Patent Application Publication
(10) Pub. No.: US 2024/0101600 A1
(43) Pub. Date: Mar. 28, 2024

(54) FIBROBLAST GROWTH FACTOR RECEPTOR (FGFR)-TARGETING ANTAGONISTIC SHORT PEPTIDE

(71) Applicant: WENZHOU MEDICAL UNIVERSITY, Wenzhou (CN)

(72) Inventors: Xiaokun Li, Wenzhou (CN); Jianzhang Wu, Wenzhou (CN); Wulan Li, Wenzhou (CN); Lingzi Chen, Wenzhou (CN); Lei Fan, Wenzhou (CN); Yuepiao Cai, Wenzhou (CN)

(21) Appl. No.: 17/768,296

(22) PCT Filed: Dec. 8, 2020
(86) PCT No.: PCT/CN2020/134550

§ 371 Date: Apr. 12, 2022

(30) Foreign Application Priority Data
Dec. 10, 2019 (CN) 201911254125.6

(51) Int. Cl. C07K 7/06

(52) U.S. Cl. CPC C07K 7/06; A61K 38/00

(57) ABSTRACT

The present disclosure discloses a fibroblast growth factor receptor (FGFR)-targeting antagonistic short peptide, namely a short peptide compound P48 with the amino acid sequence of Ser-Pro-Pro-Arg-Tyr-Pro-Gly-Gly-Gly-Ser-NH₂, where the short peptide compound P48 inhibits an FGFR pathway to inhibit cell proliferation and migration.

### TECHNICAL FIELD

The present disclosure relates to the technical field of short peptide pharmacology, in particular to an antagonistic short peptide targeting fibroblast growth factor receptor (FGFR).

### BACKGROUND

Fibroblast growth factor receptor (FGFR) drives important developmental signal transduction pathways that affect cell proliferation, migration and survival. Abnormal FGF signaling involves many cancers (Turner, N. and Grose, R. (2010) Nat. Rev. Cancer 10: 116-29). An FGFR family includes FGFR1, FGFR2, FGFR3 and FGFR4. The FGFR is a tyrosine kinase activated by gene amplification and mutation, or chromosomal translocation or rearrangement in some tumors. For example, FGFR1 amplification was found in squamous cell lung cancer and estrogen receptor-positive breast cancer, FGFR2 amplification was found in gastric cancer and breast cancer, FGFR2 mutation was observed in endometrial cancer, and FGFR3 mutation was observed in bladder cancer. FGFR fusion genes have also been reported in various blood and solid tumor cancers. For example, FGFR1-ERLIN2 in breast cancer, FGFR2-KIAA1967 in squamous cell lung cancer, and FGFR3 translocation (4, 14) in multiple myeloma were found. It is noted that some fusions occur in different cancers. For example, FGFR3-TACC3 fusion occurs in glioblastoma, bladder cancer and squamous cell carcinoma. Currently, no FGFR inhibitor has been approved for marketing, such that it is of important research value to find effective FGFR-targeting inhibitors.

Targeted inhibitors mainly include small molecule inhibitors, antibody drugs, peptide inhibitors and derivatives thereof. Compared with the small molecule inhibitors, the peptide inhibitors have high affinity and specificity, and relatively minor adverse reactions. Compared with the antibody drugs, the peptide inhibitors have a low molecular weight and a stronger tissue penetration activity. In view of the above advantages, the peptide inhibitors are favored by more and more scholars. By 2015, more than 60 peptide inhibitors have been approved for marketing worldwide and more than 140 peptide inhibitors are under clinical research. However, researches on FGFR antagonistic peptides are still at the stage of preclinical research.

The researches on the FGFR antagonistic peptides are mainly dragged by easy degradation and short half-life.

### SUMMARY

In view of the shortcomings of the prior art, an objective of the present disclosure is to provide an FGFR-targeting antagonistic short peptide. A short peptide compound P48 can effectively target and inhibit FGFR1, FGFR2 and FGFR3, and exhibits a desirable anti-tumor activity in vivo and in vitro.

In order to achieve the above goal, the present disclosure provides the following technical solution: an FGFR-targeting antagonistic short peptide, including a short peptide compound P48 capable of targeting and binding to an extramembrane immunoglobulin domain of the FGFR and has the amino acid sequence of Ser-Pro-Pro-Arg-Tyr-Pro-Gly-Gly-Gly-Ser-NH₂, where the FGFR is at least one of FGFR1, FGFR2 and FGFR3.

In the present disclosure, the short peptide compound P48 may target and bind to an extramembrane immunoglobulin domain of the FGFR.

Use of the FGFR-targeting antagonistic short peptide as an active ingredient in a drug is provided.

In the present disclosure, the drug may be used to treat diseases related to FGFR disorders by antagonizing a signal pathway of at least one of FGFR1, FGFR2 and FGFR3 in body.

In the present disclosure, the FGFR disorders may include an expression level of at least one of FGFR1, FGFR2 and FGFR3 exceeding a predetermined threshold level and a mutation of at least one of FGFR1, FGFR2 and FGFR3.

In the present disclosure, the drug may be used to treat tumors caused by disorders of at least one of FGFR1, FGFR2 and FGFR3.

In the present disclosure, the short peptide compound P48 in the FGFR-targeting antagonistic short peptide may inhibit growth and invasion of tumor cells by inhibiting at least one of an FGFR1 pathway, an FGFR2 pathway and an FGFR3 pathway.

A drug for treating diseases related to FGFR disorders includes the FGFR-targeting antagonistic short peptide, or a pharmaceutically acceptable salt or ester thereof.

In the present disclosure, the drug may further include a pharmaceutically acceptable excipient.

In the present disclosure, a dosage form of the drug may be an injection, a tablet, a capsule, an aerosol, a suppository, a film, a controlled release, a sustained release or a nano preparation.

Also provided is a nucleic acid molecule, including a nucleic acid sequence encoding a pre-amino-modified FGFR-targeting antagonistic short peptide (i.e. FGFR-targeting antagonistic short peptide without subject to amino modification), where the pre-amino-modified FGFR-targeting antagonistic short peptide has a sequence of Ser-Pro-Pro-Arg-Tyr-Pro-Gly-Gly-Gly-Ser.

Also provided is a vector including the nucleic acid molecule.

Also provided is a recombinant cell including the vector.

The present disclosure further provides is a preparation method for the FGFR-targeting antagonistic short peptide, including the following steps: culturing the recombinant cell, and purifying the culture to obtain a pre-amino-modified FGFR-targeting antagonistic short peptide, where the pre-amino-modified FGFR-targeting antagonistic short peptide has a sequence of Ser-Pro-Pro-Arg-Tyr-Pro-Gly-Gly-Gly-Ser; and subjecting the pre-amino-modified FGFR-targeting antagonistic short peptide to amino modification at an end of the pre-amino-modified FGFR-targeting antagonistic short peptide to obtain the FGFR-targeting antagonistic short peptide according to claim 1.

Use of the FGFR-targeting antagonistic short peptide as a diagnostic reagent is provided.

The present disclosure further provides a method for diagnosing diseases related to FGFR disorders, including using the FGFR-targeting antagonistic short peptide, where the FGFR disorders include an expression level of at least one of FGFR1, FGFR2 and FGFR3 exceeding a predetermined threshold level and a mutation of at least one of FGFR1, FGFR2 and FGFR3.

The present disclosure further provides a method for preventing and/or treating diseases related to FGFR disorders includes administering the drug as defined above, where the FGFR disorders include expression level of at least one of FGFR1, FGFR2 and FGFR3 exceeding a predetermined threshold level and a mutation of at least one of FGFR1, FGFR2 and FGFR3.

The advantages of the present disclosure are as follows: compared with the prior art, the short peptide compound P48 obtained through unremitting efforts has a stable secondary structure and a relatively long half-life. In addition, the short peptide compound P48 can effectively target and inhibit the FGFR, and exhibits a desirable anti-tumor activity in vivo and in vitro, which is expected to be a candidate peptide inhibitor-based drug for cancer treatment.

The present disclosure will be described in more detail hereinafter with reference to the accompanying drawings and specific examples.

### BRIEF DESCRIPTION OF THE DRAWINGS

To describe the technical solutions in the embodiments of the present disclosure or in the prior art more clearly, the accompanying drawings required for describing the embodiments or the prior art will be described briefly below. Apparently, the accompanying drawings in the following description show some embodiments of the present disclosure, and a person of ordinary skill in the art may still derive other drawings from these accompanying drawings without creative efforts.

FIG. 1 shows a schematic diagram of analysis of the structure, stability and ability to bind to FGFR1 of a short peptide compound P48 in an embodiment of the present disclosure.

FIG. 2 shows the ability of the short peptide compound P48 in an embodiment of the present disclosure to bind to FGFR1C (FIG. 2A), FGFR2B (FIG. 2B) and FGFR3B (FIG. 2C).

FIG. 3 shows a schematic diagram showing detection of an inhibitory activity of the short peptide compound P48 in an embodiment of the present disclosure on FGFR1 signaling pathway in highly-transformed human embryonic kidney cells HEK-293, fibroblasts MEF-WT and Balb/c 3T3.

FIG. 4 shows a schematic diagram of the inhibitory activity of the short peptide compound P48 in an embodiment of the present disclosure on the FGFR1 signaling pathway in various tumor cell lines.

FIG. 5 shows a schematic diagram of the in vitro anti-tumor activity of the short peptide compound P48 in an embodiment of the present disclosure.

FIG. 6 shows a schematic diagram of the in vivo anti-tumor activity of the short peptide compound P48 in an embodiment of the present disclosure.

### DETAILED DESCRIPTION OF THE EMBODIMENTS

To make the objectives, technical solutions, and advantages of the present disclosure clearer, the present disclosure will be further described in detail with reference to the accompanying drawings.

The present disclosure will be described in detail below with reference to the examples. These examples are for illustrative purposes only and are not intended to limit the protection scope of the present disclosure.

The present disclosure provides an FGFR-targeting antagonistic short peptide including a short peptide compound P48 capable of targeting and binding to an extramembrane immunoglobulin domain of the FGFR and has the amino acid sequence of:

Ser-Pro-Pro-Arg-Tyr-Pro-Gly-Gly-Gly-Ser-NH₂, where the FGFR is at least one of FGFR1, FGFR2 and FGFR3.

In the present disclosure, the short peptide compound P48 is capable of targeting and binding to the extramembrane immunoglobulin domain of FGFR1. The short peptide compound P48 has an ability to bind to FGFR1 significantly better than that to FGFR2 and FGFR3. Further, the short peptide compound P48 has the best ability to bind FGFR1C.

Use of the FGFR-targeting antagonistic short peptide as an active ingredient in a drug is provided.

In the present disclosure, the drug is used to treat diseases related to FGFR disorders by antagonizing a signal pathway of at least one of FGFR1, FGFR2 and FGFR3 in a body.

In the present disclosure, the FGFR disorders includes an expression level of at least one of FGFR1, FGFR2 and FGFR3 exceeding a predetermined threshold level and a mutation of at least one of FGFR1, FGFR2 and FGFR3. The expression level of at least one of FGFR1, FGFR2 and FGFR3 exceeding a predetermined threshold level refers to at least one of FGFR1, FGFR2 and FGFR3 has a high expression level.

In the present disclosure, the drug is used to treat tumors caused by disorders of at least one of FGFR1, FGFR2 and FGFR3.

In the present disclosure, the short peptide compound P48 in the FGFR-targeting antagonistic short peptide inhibits growth and invasion of tumor cells by inhibiting at least one of an FGFR1 pathway, an FGFR2 pathway and an FGFR3 pathway.

Preferably, the short peptide compound P48 has a stable secondary structure and a relatively prolonged half-life, and has a significant specificity for target selection. Therefore, the short peptide compound P48 has excellent medical and market prospects as a targeted drug.

A drug for treating diseases related to FGFR disorders includes an antagonistic short peptide targeting at least one of FGFR1, FGFR2 and FGFR3, or a pharmaceutically acceptable salt or ester thereof.

Further, the drug further includes a pharmaceutically acceptable excipient.

Further, a dosage form of the drug is an injection, a tablet, a capsule, an aerosol, a suppository, a film, a controlled release, a sustained release or a nano preparation.

In the present disclosure, the provided FGFR-targeting antagonistic short peptide can be artificially synthesized by well-known techniques for synthesizing polypeptides; alternatively, a nucleic acid sequence of the pre-amino-modified FGFR-targeting antagonistic short peptide (FGFR-targeting antagonistic short peptide without amino modification) can be obtained using standard recombinant DNA procedures for cloning, encoding and expressing, where the pre-amino-modified FGFR-targeting antagonistic short peptide has a sequence of Ser-Pro-Pro-Arg-Tyr-Pro-Gly-Gly-Gly-Ser.

The present disclosure further provides a nucleic acid molecule including a nucleic acid sequence encoding a short peptide before an amino modification of a nucleic acid sequence of the FGFR-targeting antagonistic short peptide, where the pre-amino-modified FGFR-targeting antagonistic short peptide has a sequence of Ser-Pro-Pro-Arg-Tyr-Pro-Gly-Gly-Gly-Ser. The nucleic acid includes single-stranded or double-stranded DNAs or RNAs. The nucleic acid can be operably ligated to an expression control sequence, that is, to a sequence necessary to express the sequence for encoding nucleic acid. Such expression control sequences may include promoters, enhancers, ribosome binding sites and/or transcription termination sequences.

A vector includes the nucleic acid. The vector may be a plasmid, a phagemid, a phage, a viral vector or a retroviral vector. The nucleic acid sequence encoding the pre-amino-modified FGFR-targeting antagonistic short peptide is inserted into the vector. The vector may additionally contain an origin of replication and/or a selectable marker gene.

The nucleic acid molecule can be ligated to a vector containing a selection marker for reproduction in a host. Generally, the plasmid vector is introduced into a precipitate such as a calcium phosphate precipitate or a rubidium chloride precipitate, or into a complex with charged lipids, or into carbon-based atomic clusters (such as fullerenes). If the vector is a virus, the plasmid vector can be packaged in vitro with an appropriate packaging cell line before being applied to the host cell.

In the present disclosure, the vector is an expression vector, where nucleic acid molecules are operably ligated to one or more control sequences, thereby being allowed transcription and optional expression in prokaryotic and/or eukaryotic host cells. An expression of the nucleic acid molecules includes transcripting the nucleic acid molecules into preferably translatable mRNA. Regulatory elements that ensure expression in eukaryotic cells, preferably mammalian cells, are well known to those skilled in the art. These regulatory elements usually contain regulatory sequences to ensure transcription initiation and optional polyadenylic acid signals to ensure transcription termination and transcription stability. Other regulatory elements may include transcription enhancers and translation enhancers. Possible regulatory elements that allow expression in prokaryotic host cells include, for example, lac, trp or tac promoters in E. coli. In addition, regulatory elements that allow expression in eukaryotic host cells include an alcohol oxidase 1 (AOX1) promoter or a galactose 1 (GAL-1) promoter in yeasts, or a cytomegalovirus (CMV)-promoter, a simian virus 40 (SV40)-promoter, a Rous sarcoma virus (RSV)-promoter, a CMV-enhancer, an SV40-enhancer or a globulin intron in mammalian and other animal cells. In addition to elements responsible for transcription initiation, such regulatory elements may also include transcription termination signals downstream of polynucleotides, such as SV40-polyadenylation sites or tk-polyadenylation sites. In this case, suitable expression vectors are known in the art. Preferably, the vector is an expression vector and/or a gene transfer or targeting vector. Expression vectors derived from viruses such as retroviruses, vaccinia viruses, adeno-associated viruses, herpes viruses, or bovine papilloma viruses can be used to deliver the polynucleotides or vectors of the present disclosure to target cell clusters. The recombinant viral vector can be constructed using methods well known to those skilled in the art. Alternatively, the nucleic acid molecules of the present disclosure can be reconstituted into liposomes for delivery to target cells. In addition, the present disclosure relates to a host including the nucleic acid molecule or the vector. The nucleic acid molecule or the vector can be introduced into the host by transformation, transfection or transduction according to any method known in the art.

A recombinant cell that is a host cell carrying the nucleic acid molecule or the vector is disclosed, where the host can be any prokaryotic or eukaryotic cells, such as cells of bacteria, insects, fungi, plants, animals, mammals or preferably humans. Preferably, the cells of fungi are, for example, those of Saccharomyces, especially those of S. cerevisiae. The term “prokaryotic” refers to all bacteria that can be transformed or transfected with polynucleotides to express the variant polypeptides of the present disclosure. Prokaryotic hosts may include Gram-negative and Gram-positive bacteria, such as E. coli, Salmonella typhimurium, Serratia marcescens and Bacillus subtilis. The polynucleotides encoding mutant forms of the variant polypeptides of the present disclosure can be used to transform or transfect a host using any technique generally known to those of ordinary skill in the art. It is well known in the art that there are methods for preparing fused and operably-ligated genes and expressing the genes in bacteria or animal cells. The genetic constructs and methods described herein can be used to express the variant antibodies, antibody fragments or derivatives thereof of the present disclosure in, for example, a prokaryotic host. Generally, expression vector containing a promoter sequence is used in combination with the host, where the promoter sequence promotes efficient transcription of an inserted nucleic acid molecule. The expression vector usually contains an origin of replication, a promoter and a terminator, and specific genes that can provide phenotypic selection for transformed cells. The transformed prokaryotic host can be grown in a fermenter and cultured according to techniques known in the art to achieve optimal cell growth. Then, the antibodies, antibody fragments or derivatives thereof of the present disclosure can be isolated from a growth medium, a cell lysate or a cell membrane fraction. In the present disclosure, isolation and purification can be conducted by any conventional means for the antibodies, the antibody fragments or the derivatives thereof expressed by microorganisms or other methods. For example, preparative chromatography-based separation and immunological separation can be used, such as those methods involving the use of monoclonal or polyclonal antibodies.

A preparation method of the FGFR-targeting antagonistic short peptide is provide, which includes culturing the recombinant cell, and purifying the culture to obtain the FGFR-targeting antagonistic short peptide.

Use of the FGFR-targeting antagonistic short peptide as a diagnostic reagent is provided. The use of the diagnostic reagent specifically includes: detecting the expression level of at least one of FGFR1, FGFR2 and FGFR3 by the FGFR-targeting antagonistic short peptide.

A method for diagnosing diseases related to FGFR disorders includes using the FGFR-targeting antagonistic short peptide, where the FGFR disorders include an expression level of at least one of FGFR1, FGFR2 and FGFR3 exceeding a predetermined threshold level and a mutation of at least one of FGFR1, FGFR2 and FGFR3. The method includes: contacting the antibody of the present disclosure with a cell or a tissue suspected of carrying one or more of FGFR1, FGFR2 and FGFR3 on a surface. A suitable method for detecting FGFR4 expression in a sample can be enzyme-linked immunosorbent assay (ELISA) or immunohistochemistry (IHC).

A method for preventing and/or treating diseases related to FGFR disorders includes administering the drug, where the FGFR disorders include an expression level of at least one of FGFR1, FGFR2 and FGFR3 exceeding a predetermined threshold level and a mutation of at least one of FGFR1, FGFR2 and FGFR3.

Specifically, the FGFR1-targeting antagonistic short peptide has the following amino acid sequence (Example 1):

Ser-Pro-Pro-Arg-Tyr-Pro-Gly-Gly-Gly-Ser-NH₂.

Experimental results show that, compared with a precursor peptide P9, the short peptide compound P48 has a stable secondary structure and a relatively prolonged half-life. In addition, molecular docking experiments have found that the short peptide compound P48 can form a stable hydrogen bond with FGFR1. The surface plasmon resonance (SPR) experiment has found that P48 can competitively bind to FGFR1 with basic fibroblast growth factor (bFGF), and a higher concentration leads to a stronger binding force (Example 1).

Meanwhile, Western blot experiments show that in highly-transformed human embryonic kidney cells HEK-293, fibroblasts MEF-WT and Balb/c 3T3, the short peptide compound P48 can inhibit activation of FGFR1 induced by bFGF in a concentration-dependent manner (Example 2). In addition, the short peptide compound P48 has an inhibitory effect on FGFR1 signals similar to that of selective serotonin reuptake (SSR), and has no significant inhibitory effect on downstream phospholipase Cy (PLCy) (Example 2). Wild-type and FGFR1-FGFR2-FRS2a knock-out MEF cells are subjected to flow cytometry, and it is shown that the short peptide compound P48 only has a binding effect on the wild-type MEF cells (Example 2).

Various tumor cell lines with FGFR1 disorders are subjected to Western blot experiments, and results show that the short peptide compound P48 can inhibit the FGFR1 signaling pathway of cervical cancer, melanoma, lung cancer and gastric cancer in a concentration-dependent manner (Example 3). In addition, in gastric cancer cell lines, the short peptide compound P48 has an inhibitory effect on FGFR1 signaling comparable to that of the SSR, and has an activity weaker than that of AZD4547. This may be related to different binding types, that is, the short peptide compound P48 may be similar to the SSR as an extramembrane inhibitor, while the AZD4547 is an intra-membrane inhibitor. Flow cytometry experiments have further found that the short peptide compound P48 shows a desirable binding effect on the FGFR1 site of the cell membrane surface (Example 3). Further, the Western blot experiments have found that the short peptide compound P48 can only inhibit the activation of FGFR1 induced by bFGF, and has no effect on stimulation of acidic fibroblast growth factor (aFGF), feratinocyte growth factor (KGF) and epidermal growth factor (EGF) (Example 3).

In addition, the short peptide compound P48 can significantly inhibit the viability of cancer cells in Hela229, B16-F10, NCI-H460 and SGC-7901 cells. In addition, in gastric cancer cells SGC-7901, compared with a control group, the short peptide compound P48 can inhibit cell migration in a concentration-dependent manner (Example 4). The flow cytometry cycle experiment has found that the short peptide compound P48 can effectively block the cell cycle in a G0/G1 phase (Example 4).

Finally, in a mouse model with transplanted tumors, the short peptide compound P48 can significantly inhibit the growth of gastric cancer cells SGC-7901, and has a desirable inhibitory effect on the FGFR1 signaling pathways (including phosphorylated FLG, FRS2a and ERK1/2 protein) (Example 5). In addition, immunohistochemical experiments have found that compared with the control group, the short peptide compound P48 can inhibit the expression of Ki67 in a concentration-dependent manner (Example 5). The results indicate that the short peptide compound P48 can also inhibit the FGFR1 pathway to inhibit cell proliferation in vivo to exert a desirable anti-tumor activity (Experimental Example 5).

As mentioned above, the research results show that the short peptide compound P48 has a desirable stability and can exert an anti-tumor effect by inhibiting the FGFR1 pathway, which has a prospect of being developed as an anti-tumor drug.

Therefore, the present disclosure provides use of an efficient and stable FGFR1-targeting antagonist peptide P48 in preparation of an anti-tumor drug; the anti-tumor drug can treat tumors by selectively inhibiting the FGFR1 signaling pathway to inhibit cell proliferation and migration.

The present disclosure further provides a pharmaceutical composition for treating tumors, including a therapeutically effective amount of an active ingredient and a pharmaceutical excipient, where the active ingredient is the FGFR1-targeting antagonistic short peptide or pharmaceutically acceptable salts and pharmaceutical excipients thereof. The “pharmaceutical excipients” refer to conventional pharmaceutical carriers in the pharmaceutical field, including: diluents such as starch, sucrose, dextrin, lactose, pregelatinized starch, microcrystalline cellulose and calcium phosphate; wetting agents such as distilled water and ethanol; adhesives such as starch slurry, cellulose derivatives, povidone, gelatin, polyethylene glycol and sodium alginate solution; disintegrants such as dry starch, sodium carboxymethyl starch, low-substituted hydroxypropyl cellulose and effervescent disintegrants; lubricants such as magnesium stearate, micronized silica gel, talc, hydrogenated vegetable oil, polyethylene glycols and sodium lauryl sulfate; coloring agents such as titanium dioxide, sunset yellow FCF, methylene blue and medicinal iron oxide red. In addition, other excipients such as flavors and sweeteners can also be added to the composition.

Various dosage forms of the pharmaceutical composition of the present disclosure can be prepared according to conventional production methods in the field of pharmacy. For example, the active ingredient is mixed with one or more carriers to be made into a desired dosage form. A dosage form of the drug includes a granule, an injection, a tablet, a capsule, an aerosol, a suppository, a film, a dripping pill, an ointment, a controlled release, a sustained release or a nano preparation. The composition can be administered to patients in need of such treatment by oral, nasal inhalation, rectal or parenteral routes of administration. When used for oral administration, the composition can be made into conventional solid preparations such as tablets, powders, granules and capsules, and into liquid preparations such as water or oil suspensions or syrups and elixirs; when used for parenteral administration, the composition can be made into injection solution and water or oily suspension.

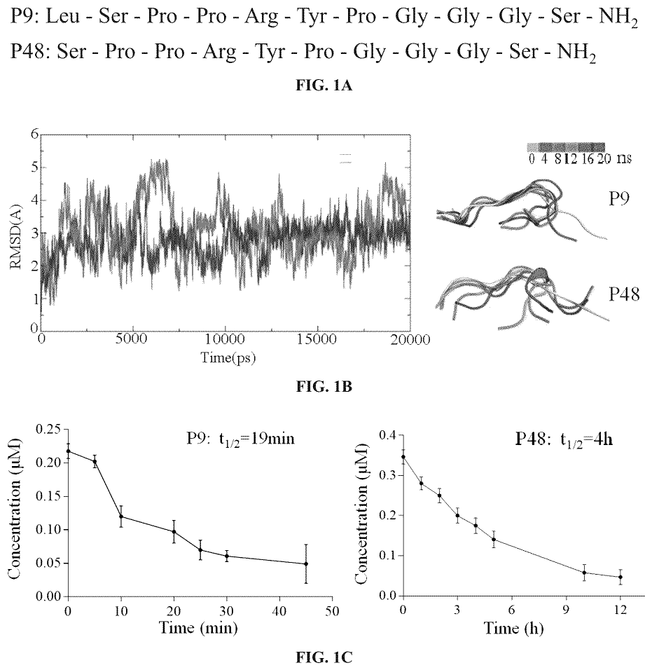

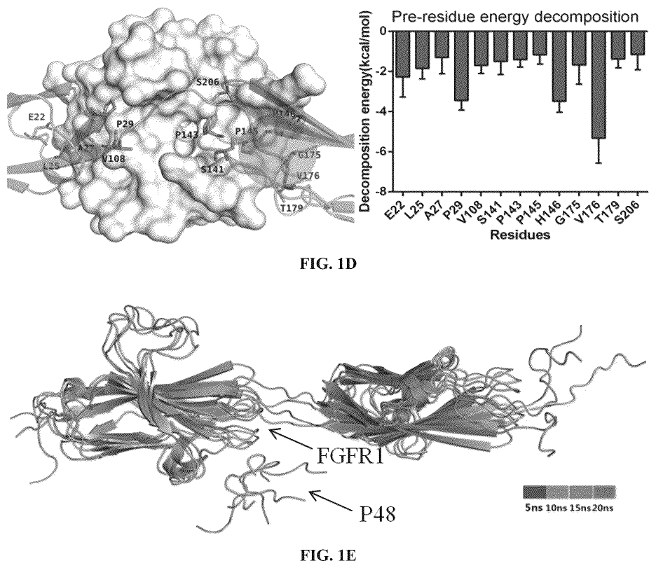

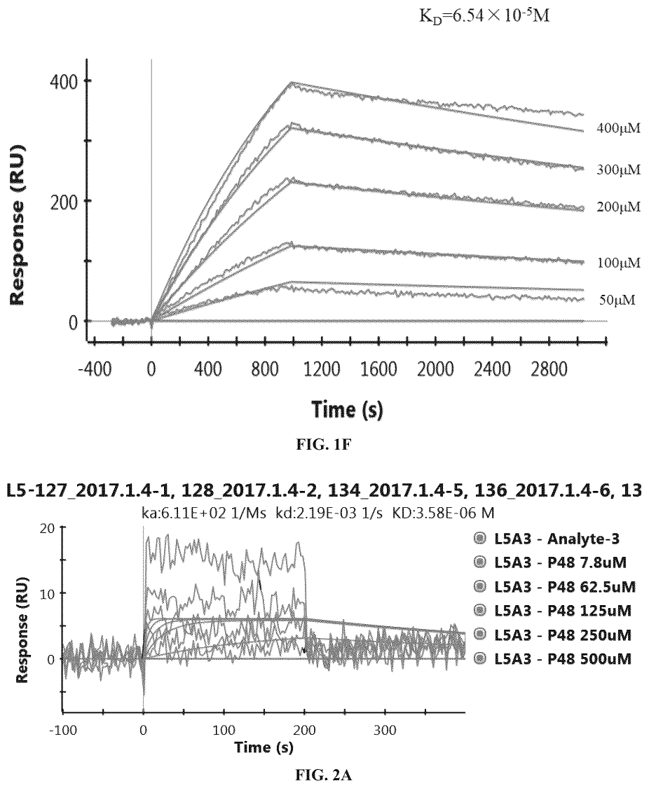

Referring to FIG. 1, analysis on a structure, stability and ability to bind to FGFR1 of a short peptide compound P48 are shown, where:

(A) sequences of a precursor peptide P9 and a degraded short peptide P48; (B) a: root-mean-square deviation (RMSD) of a C atom in the P9 and P48 main chains varies with the time of molecular dynamics simulation; and b: an initial conformation of the P9 and P48 and a superimposed image of conformations at 4 ns, 8 ns, 12 ns, 16 ns and 20 ns; (C) half-life of the P9 and P48 in human plasma; (D) a: molecular dynamics simulation and free energy calculation of a bFGF-FGFR1 complex; b: analysis of key residues to maintain stability of the bFGF-FGFR1 complex; (E) molecular docking of FGFR1B and P48; (F) SPR detection of interaction between FGFR1B and P48;

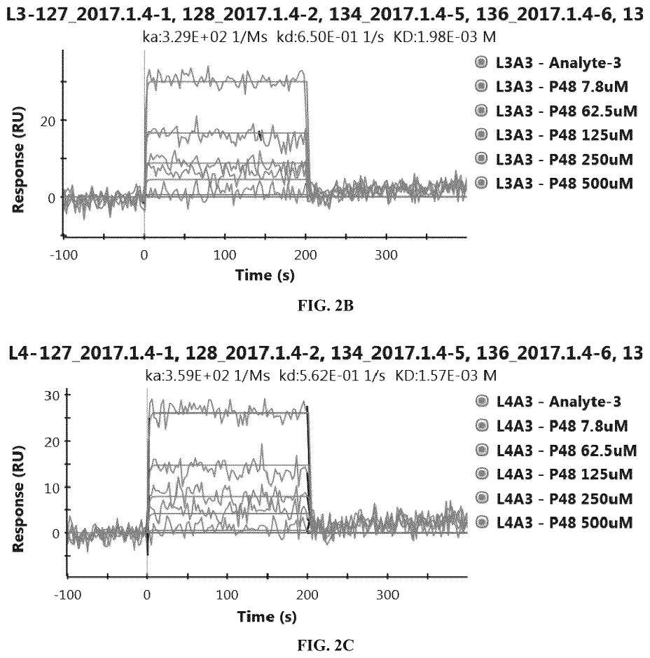

FIG. 2: (A) SPR detection of interaction between FGFR1C and P48; (B) SPR detection of interaction between FGFR2B and P48; (C) SPR detection of interaction between FGFR3B and P48;

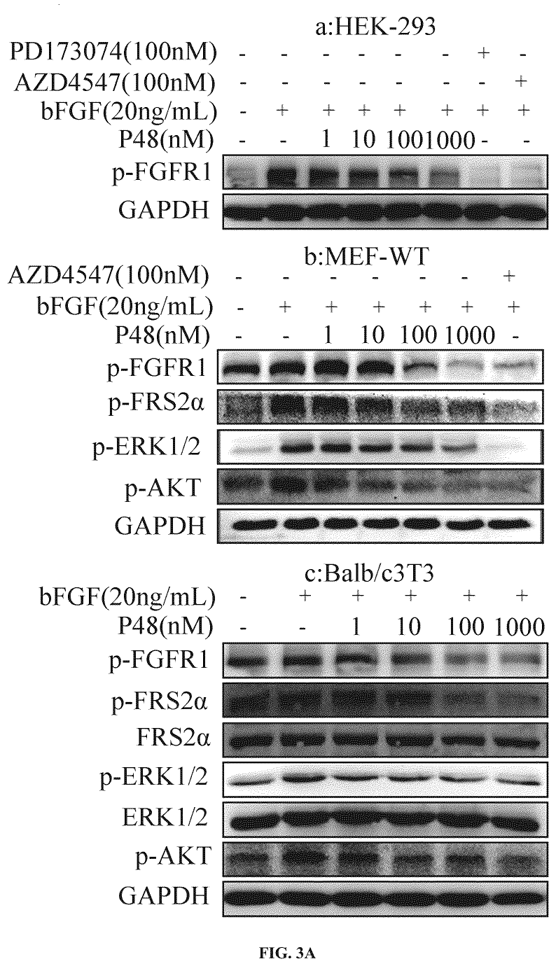

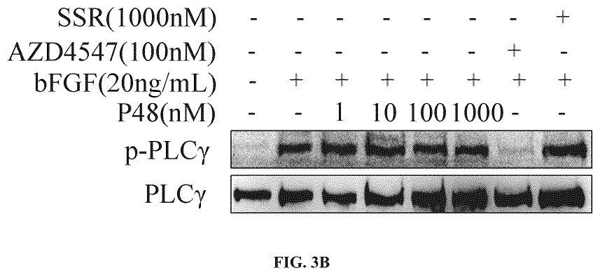

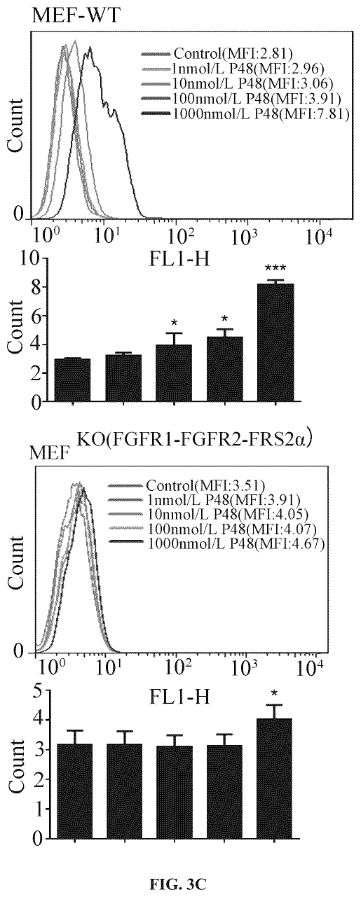

FIG. 3 shows the detection of an inhibitory activity of P48 on an FGFR1 signaling pathway in highly-transformed human embryonic kidney cells HEK-293, fibroblasts MEF-WT and Balb/c 3T3, where:

(A) after pretreatment with the short peptide compound P48 for 10 min or with AZD4547/PD173074 for 2 h, incubation is conducted with bFGF for 15 min to detect inhibition of drugs on FGFR1 and downstream proteins thereof in HEK-293, MEF-WT and Balb/c3T3 cells; (A) after 10 min of P48 pretreatment or 2 h of AZD4547/SSR treatment, bFGF stimulation is conducted for 15 min to detect inhibition of drugs on PLCy; (C) flow cytometry analysis of an ability of P48 to bind to FGFR1 in MEF-WT and FGFR1-FGFR2-FRS2α knockout MEF cells; *, p<0.05; ***, p<0.001, vs Control;

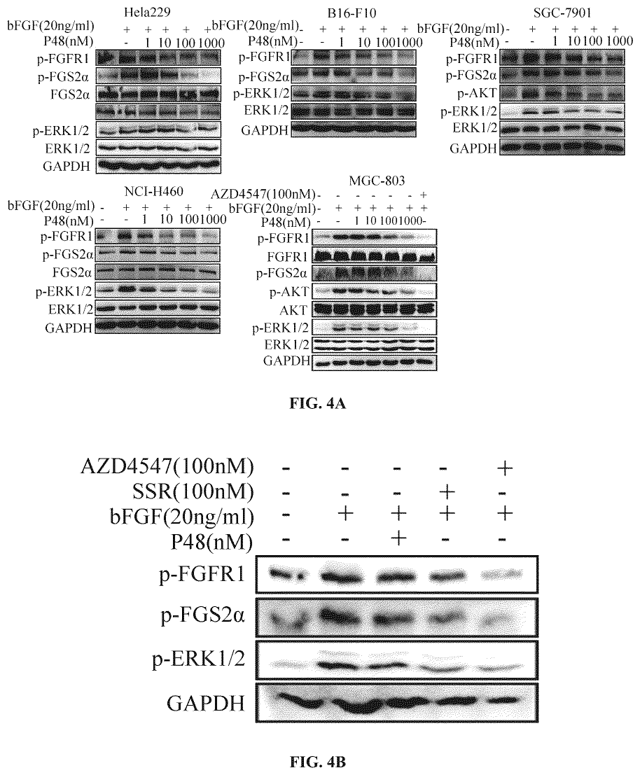

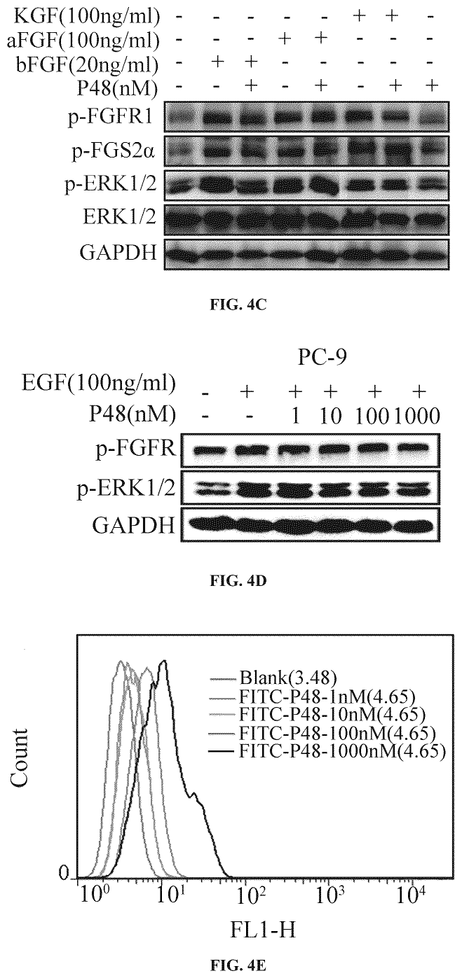

FIG. 4: an inhibitory activity of the short peptide compound P48 on the FGFR1 signaling pathway in various tumor cell lines, where:

(A) after pretreatment with the short peptide compound P48 for 10 min, incubation is conducted with bFGF for 15 min to detect inhibition of drugs on FGFR1 and downstream proteins thereof; (B) after pretreatment with P48 for 10 min or with AZD4547/SSR for 2 h, incubation is conducted with bFGF for 15 min to detect an inhibitory activity of drugs on phosphorylated FGFR1 and downstream proteins thereof in SGC-7901 cells; (C) after bFGF/aFGF stimulation for 15 min or KGF stimulation for 20 min, the inhibitory activity of the short peptide compound P48 is detected on phosphorylated FGFR1 and downstream proteins thereof in SGC-7901 cells; (D) after EGF stimulation for 10 min, the inhibitory activity of the short peptide compound P48 is detected on phosphorylated EGFR and ERK1/2 in PC-9 cells; (E) flow cytometry detection of binding ability of short peptide compound P48 with FGFR1 in SGC-7901 cells;

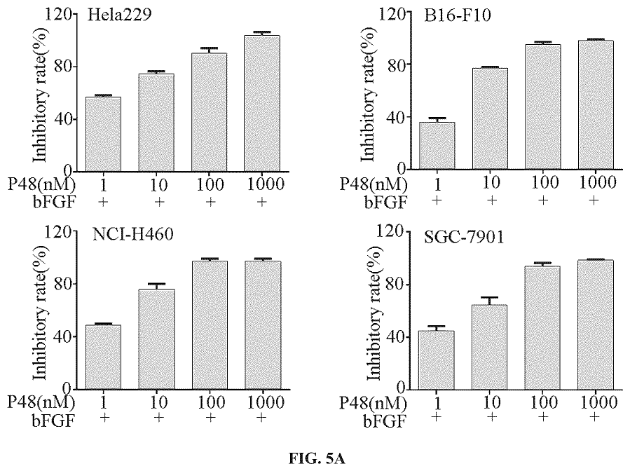

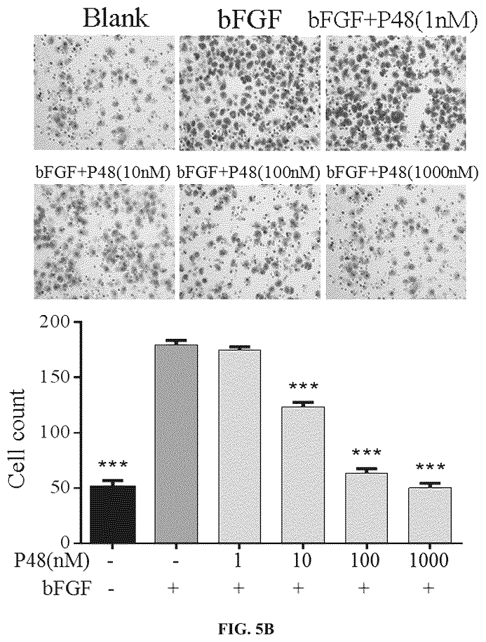

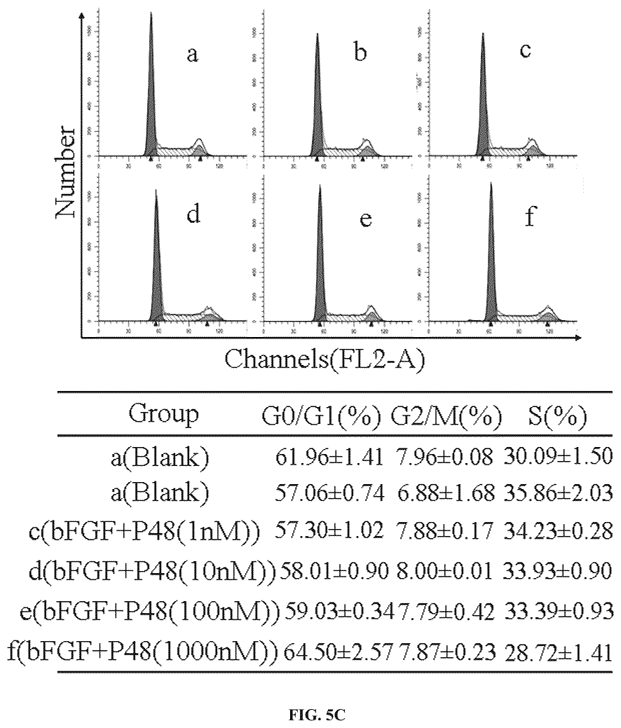

FIG. 5 shows the in vitro anti-tumor activity of the short peptide compound P48, where: (A) detection of growth inhibitory effect of the short peptide compound P48 on cancer cells using a MTT method; (B) detection of inhibitory activity of the short peptide compound P48 on migration of SGC-7901 cells by cell invasion experiment; (C) flow cytometry analysis of the short peptide compound P48 on cell cycle arrest; ***, p<0.001, vs bFGF; and

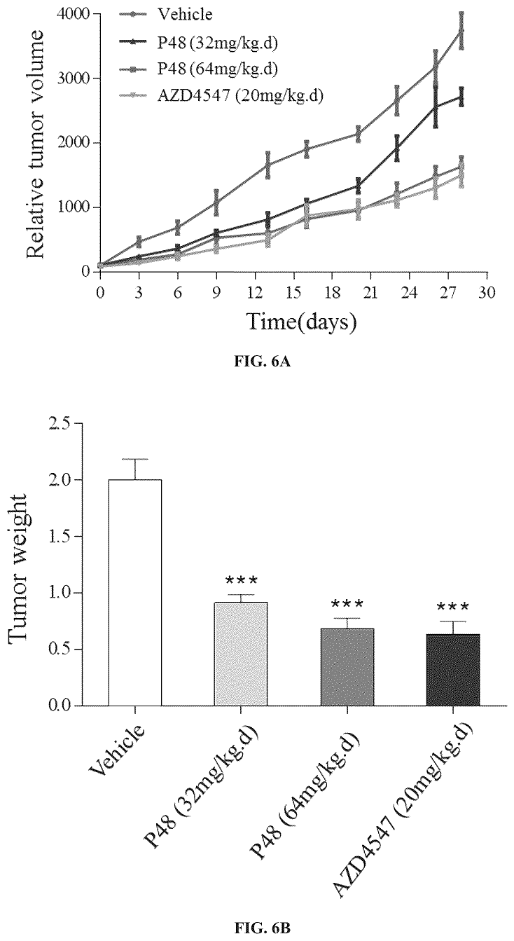

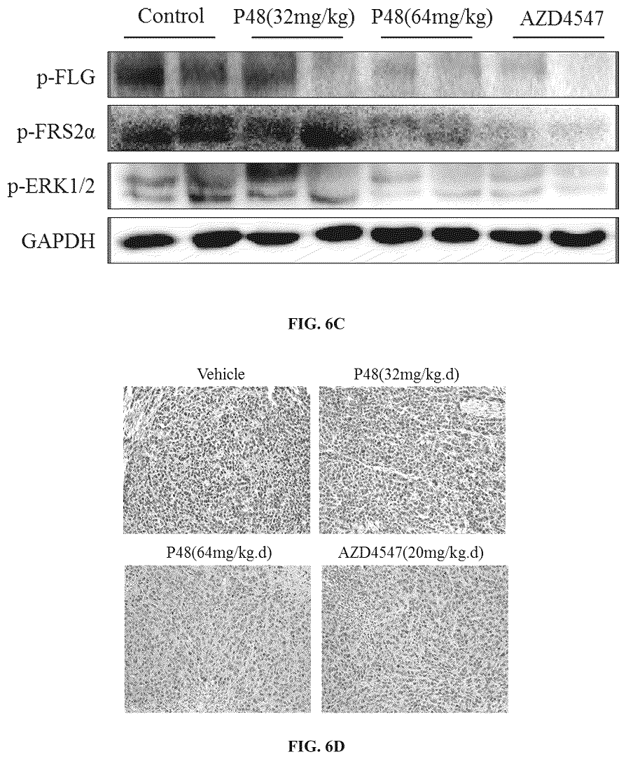

FIG. 6 shows the in vivo anti-tumor activity of the short peptide compound P48, where: (A) tumor volume measurement in a control group and a treatment group; (B) tumor weighing; (C) inhibitory effect of the short peptide compound P48 on phosphorylation level of FLG and downstream proteins thereof in nude mice; and (D) immunohistochemical detection of inhibitory activity of the short peptide compound P48 on pro-proliferation protein Ki67.

### Example 1

Analysis of spatial structure, stability and FGFR1 binding activity of a short peptide compound P48:

Amino acid sequence of P9 degraded short peptide P48 was analyzed using Amber11 program. Results are shown in FIG. 1A. An initial structure of the P9 and P48 peptide chains was constructed using a sequence command in a Tleap module of the Amber11 program. Hydrogen atoms were added using the Tleap module of the Amber11 program, a truncated tetrahedral TIP3P solvent box was added at a distance of 10 Å from the peptide chain, and counter ions were added to the two systems to keep the system electrically neutral. The system was subjected to 20-ns NPT system simulation to obtain an optimal spatial structure. The results are shown in FIG. 1B. FIG. 1C shows the half-life of P9 and P48 in plasma. The experimental procedure was as follows: the plasma was centrifuged and the supernatant was removed, and 60 μl of peptide was added to incubate for different lengths of time (P9 was incubated for 5, 10, 15, 20, 25, 30 and 45 min; P48 was incubated for 1, 2, 3, 4, 5, 10 and 12 h), and the incubation was terminated with 200 μl of 5% glacial acetic acid. Solid phase extraction: activation was conducted with 1.5 ml of methanol, an obtained product was washed with 1.5 ml of H₂O, 400 μl of a sample was loaded, washed with 1 ml of H₂O, and elution was conducted with 2 ml of 30% acetonitrile to collect samples, and the samples were freeze-dried and subjected to liquid chromatography analysis. FIG. 1D shows the molecular dynamics simulation of the FGF2-FGFR1 complex system to determine the key amino acid residues that maintain the binding of FGF2-FGFR1. The experimental procedure was as follows: an FGF2-FGFR1 protein crystal structure (PDB ID: 1CVS) was selected to construct an initial structure of the FGF2-FGFR1 complex system by molecular dynamics simulation. Hydrogen atoms were added using the Tleap module of the Amber11 program, a truncated tetrahedral TIP3P solvent box was added at a distance of 10 Å from the complex, and counter ions were added to the two systems to keep the system electrically neutral. The system used an Amber99SB force field, and a Sander module was used to minimize the energy of the system. The last 1 ns of trajectory obtained by molecular dynamics simulation of the system was selected to calculate the binding free energy, the molecules of the solvent of water and counter ion were deleted, and conformation was recorded every 10 ps to obtain a total of 101 structures. The binding free energy between FGF2 and FGFR1 were calculated by a molecular mechanics-generalized born surface area (MM-GBSA) method. The binding free energy and energy decomposition of amino acid residues were calculated using an MMPBA.py script provided by the Amber11. The above experimental steps could determine the FGFR1 binding sites; in Discovery Studio Visualizer 3.5, P48 was placed at the FGFR1 binding site, and a corresponding complex system file P48-FGFR1.pdb was saved as an initial structure file for the molecular dynamics simulation. FGFR1 coordinates were selected from FGFR1 in an A chain of the FGF2-FGFR1 protein crystal structure (PDB ID: 1CVS). Hydrogen atoms were added using the Tleap module of the Amber11 program, a truncated tetrahedral TIP3P solvent box was added at a distance of 10 Å from the complex, and counter ions were added to the two systems to keep the system electrically neutral. The system was subjected to 20-ns NPT system simulation. The results are shown in FIG. 1E. FIG. 1F (FGFR1B protein in FIG. 1F) shows the interaction between P48 and FGFR1. The experimental procedure was as follows: a sensor chip was washed with a 30 μl/min PBS, and activated with an activation buffer of 40 mM EDAC and 10 mM sNHS. After bFGF and FGFR1 flew into the sensor chip, P48 (50 μM, 100 μM, 200 μM, 300 μM and 400 μM) was injected into a sensor surface, and the obtained data was analyzed using BIAevaluation software.

Further, the interaction of P48 with FGFR1C, FGFR2B and FGFR3B was studied; the experimental procedure was the same as above. The experimental results are shown in FIG. 2, and it can be seen that P48 can be bound with FGFR1C (FIG. 2A), FGFR2B (FIG. 2B), and FGFR3B (FIG. 2C) to a certain extent. Combined with the analysis of FIG. 1F, it can be seen that the binding degree of P48 with the FGFR1 is stronger than that with FGFR2 and FGFR3, where FGFR1C has the strongest binding degree.

### Example 2

An inhibitory activity of a short peptide compound P48 on a FGFR1 signaling pathway in highly-transformed human embryonic kidney cells and fibroblasts:

Human embryonic kidney cells HEK-293, fibroblasts MEF-WT and Balb/c 3T3 were inoculated into 6-well plates, respectively. After growing adherently overnight, the cells were pretreated with P48 for 10 min or with AZD4547/PD173074/SSR for 2 h, and incubated with bFGF for 15 min. FIG. 3A shows inhibition of FGFR1 and downstream proteins thereof (FRS2a, ERK1/2 and AKT) in the HEK-293, the MEF-WT and the Balb/c3T3 cells by P48/AZD4547/PD173074. FIG. 3B shows inhibition of P48/AZD4547/SSR on FGFR1 downstream protein PLCy in MEF-WT cells. FIG. 3C shows an ability of P48 to bind to the FGFR1 in MEF-WT and FGFR1-FGFR2-FRS2a knockout MEF cells. The experimental procedure was as follows: MEF-WT and MEFKO (FGFR1-FGFR2-FRS2a) cells (1×10 6 cells/ml) were inoculated in a 35 mm petri dish. After being adhered to the wall of petri dish overnight, the cells were washed with PBS; FITC-conjugated P48 (1, 10, 100 and 1000 nM) was added, and the cells were incubated in an incubator for 30 min. The cells were immobilized with 1% paraformaldehyde in the dark and washed with PBS. An expression level of the FITC was measured at a fluorescence channel 1 (FL-1) (530 nm), with an excitation wavelength of 488 nm. Data analysis was conducted using CellQuest software (Becton Dickinson).

### Example 3

Inhibitory Activity of a Short Peptide Compound P48 on an FGFR1 Signaling Pathway in Various Tumor Cells

Cervical cancer cells Hela229, melanoma B16-F10, lung cancer cells NCI-H460, gastric cancer cells SGC-7901 and MGC-803 were inoculated into 6-well plates, respectively. After growing adherently overnight, the cells were pretreated with P48 for 10 min or with AZD4547/SSR for 2 h, and incubated with bFGF for 15 min or with KGF for 20 min. FIG. 4A shows an inhibition of FGFR1 and downstream proteins thereof (FRS2a, ERK1/2 and AKT) in the Hela229, the B16-F10, the NCI-H460, the MGC-803 and the SGC-7901 cells by P48/AZD4547 under bFGF stimulation. FIG. 4B shows an inhibition of FGFR1 and downstream proteins thereof (FRS2a and ERK1/2) in SGC-7901 cells by P48/AZD4547/SSR under bFGF stimulation. FIG. 4C shows an inhibition of FGFR1 and downstream proteins thereof (FRS2a and ERK1/2) in SGC-7901 cells by P48 under bFGF/aFGF/KGF stimulation. FIG. 4D shows an inhibition of EGFR and ERK1/2 in PC-9 cells by P48 under EGF stimulation. FIG. 4E shows binding of P48 to FGFR1 in SGC-7901 cells, where the experimental procedure was the same as that of FIG. 2C.

### Example 4

In vitro anti-tumor activity of a short peptide compound P48 Hela229 (1500 cells/100 μl), B16-F10 (1000 cells/100 μl), NCI-H460 (4000 cells/100 μl) and SGC7901 (4000 cells/100 μl) cells were inoculated in 96-well plates. After incubated overnight adherently, the original medium was replaced with a Dulbecco's modified eagle medium (DMEM) containing 0.1% fetal bovine serum (FBS) or a Roswell Park Memorial Institute 1640 (RPMI-1640) medium containing 0.1% FBS, and culture was continued for 24 h. A mixture of P48 and bFGF, or bFGF alone were added into corresponding wells, and incubated for 48 h; an MTT solution (5 mg/mL) prepared by PBS was added to the cells in each well at 37° C., and treated for 4 h. The MTT was removed by aspiration, 150 μL of DMSO was added to each well, shaking was conducted for 10 min, and an optical density (OD) value was quantified with a microplate reader (SpectraMax M2/M2e, Molecular Devices, Sunnyvale, USA) at a wavelength of 490 nm. The percentage of drug growth inhibition rate was calculated by comparing with the bFGF group. The results are shown in FIG. 5A. FIG. 5B shows that P48 can inhibit the migration of gastric cancer cells SGC-7901 in a concentration-dependent manner. The experimental procedure was as follows: 10% FBS-containing medium (500 μl) was added to a lower surface of a transwell insertion chamber, the cells together with the drug were inoculated into an upper chamber, and cultured with a serum-free medium (200 μl). After culturing in an incubator for 24 h, the cells in the lower chamber were immobilized with pre-chilled methanol and stained with crystal violet; meanwhile, the cells in the upper chamber were removed with a cotton swab. The migrating cells were captured under a light microscope. The results in FIG. 5C show that P48 can block gastric cancer cells SGC-7901 in G0/G1 phase in a concentration-dependent manner. The experimental procedure was as follows: SGC-7901 cells (6×10⁵ cells/well) were seeded in a petri dish with a diameter of 60 mm. The cells were treated with a mixture of P48 and bFGF, the bFGF and a carrier (PBS) for 24 h, washed with PBS, and fixated in 75% ice-cold ethanol overnight. The cells were stained with 500 μL of propidium iodide (PI) containing ribonuclease (550825, BD Biosciences 35Clontech, San Jose, CA, USA) at 4° C. away from light for 10 min, and filtered with 200-mesh gauze. Cell cycle analysis was conducted in a FACS Calibur flow cytometer (BD Biosciences, CA).

### Example 5

In Vitro Anti-Tumor Activity of a Short Peptide Compound P48

SGC-7901 cells were injected subcutaneously into the back of BALB/c nude mice. After 10 days, the mice were randomly divided into 4 groups, each with 12 mice, and were administered intraperitoneally. An administration group included a model group (normal saline group), an AZD4547 group (20 mg/kg·d), and a P48 group (32 mg/kg·d, 64 mg/kg·d). The length and width of the tumor were measured using a vernier caliper. The tumor volume was calculated using a formula V=0.52×L×W². The weights of the mice were recorded every 3 days, and the tumor size was recorded on the day of the death of the mice. After 28 days, the nude mice were sacrificed for dissection, and tissue samples were taken out for further study. The results are shown in FIG. 6, wherein A represents the weight change of the mice, B represents the volume and size of the tumor, C represents the expression of phosphorylated FLG, FRS2α and ERK1/2 in vivo after P48 treatment, and D represents the expression of Ki67 in vivo after P48 treatment.

In summary: the short peptide compound P48 obtained through unremitting efforts has a stable secondary structure and a relatively long half-life. In addition, the short peptide compound P48 can effectively target and inhibit FGFR1, and exhibits a desirable anti-tumor activity in vivo and in vitro, which is expected to be a candidate peptide inhibitor-based drug for cancer treatment.

The above examples describe the present disclosure in detail, and are only used to further describe, but not to limit the protection scope of the present disclosure. Technical engineers in the field can make some non-essential improvements and adjustments to the present disclosure based on the content of the above solutions, which shall fall within the protection scope of the present disclosure.

1. A fibroblast growth factor receptor (FGFR)-targeting antagonistic short peptide, comprising a short peptide compound P48 capable of targeting and binding to an extramembrane immunoglobulin domain of the FGFR and has the amino acid sequence of Ser-Pro-Pro-Arg-Tyr-Pro-Gly-Gly-Gly-Ser-NH₂, wherein the FGFR is at least one of FGFR1, FGFR2 and FGFR3.

2. The FGFR-targeting antagonistic short peptide according to claim 1, wherein the short peptide compound P48 is capable of targeting and binding to an extramembrane immunoglobulin domain of the FGFR1.

3. (canceled)

4. (canceled)

5. (canceled)

6. (canceled)

7. (canceled)

8. A drug for treating diseases related to FGFR disorders, comprising the FGFR-targeting antagonistic short peptide according to claim 1, or a pharmaceutically acceptable salt or ester thereof.

9. The drug according to claim 8, further comprising a pharmaceutically acceptable excipient.

10. The drug according to claim 9, wherein a dosage form of the drug is an injection, a tablet, a capsule, an aerosol, a suppository, a film, a controlled release, a sustained release or a nano preparation.

11. A nucleic acid molecule, comprising a nucleic acid sequence encoding the pre-amino-modified FGFR-targeting antagonistic short peptide according to claim 1, wherein the pre-amino-modified FGFR-targeting antagonistic short peptide has a sequence of Ser-Pro-Pro-Arg-Tyr-Pro-Gly-Gly-Gly-Ser.

12. (canceled)

13. (canceled)

14. (canceled)

15. (canceled)

16. A method for diagnosing, preventing, and/or treating diseases related to FGFR disorders, comprising a step of administering the FGFR-targeting antagonistic short peptide according to claim 1 to a patient in need thereof, wherein the FGFR disorders comprise an expression level of at least one of FGFR1, FGFR2 and FGFR3 exceeding a predetermined threshold level and a mutation of at least one of FGFR1, FGFR2 and FGFR3.

17. (canceled)

18. A method for diagnosing, preventing and/or treating diseases related to FGFR disorders, comprising a step of administering the FGFR-targeting antagonistic short peptide according to claim 2 to a patient in need thereof, wherein the FGFR disorders comprise an expression level of at least one of FGFR1, FGFR2 and FGFR3 exceeding a predetermined threshold level and a mutation of at least one of FGFR1, FGFR2 and FGFR3.

19. A method for diagnosing, preventing and/or treating diseases related to FGFR disorders, comprising a step of administering the drug according to claim 8 to a patient in need thereof, wherein the FGFR disorders comprise an expression level of at least one of FGFR1, FGFR2 and FGFR3 exceeding a predetermined threshold level and a mutation of at least one of FGFR1, FGFR2 and FGFR3.

20. A method for diagnosing, preventing and/or treating diseases related to FGFR disorders, comprising a step of administering the drug according to claim 9 to a patient in need thereof, wherein the FGFR disorders comprise an expression level of at least one of FGFR1, FGFR2 and FGFR3 exceeding a predetermined threshold level and a mutation of at least one of FGFR1, FGFR2 and FGFR3.

21. A method for diagnosing, preventing and/or treating diseases related to FGFR disorders, comprising a step of administering the drug according to claim 10 to a patient in need thereof, wherein the FGFR disorders comprise an expression level of at least one of FGFR1, FGFR2 and FGFR3 exceeding a predetermined threshold level and a mutation of at least one of FGFR1, FGFR2 and FGFR3.


SEQUENCE LISTING (1 sequence)

SEQ ID NO: 1 - protein, 10 aa, Artificial Sequence
  FIBROBLAST GROWTH FACTOR RECEPTOR (FGFR)-TARGETING ANTAGONISTIC SHORT PEPTIDE
SGGGPYRPPS